(19) United States
(12) Patent Application Publication
(10) Pub. No.: US 2011/0179686 A1
(43) Pub. Date: Jul. 28, 2011

(54) PEDIATRIC IDENTIFICATION TAG WITH WINGS

(76) Inventors: Steve L. Morehouse, Agua Dulce, CA (US); Karen L. Joseph, Encino, CA (US); Martin E. Saenz, Tijuana (MX)

(21) Appl. No.: 12/695,094

(22) Filed: Jan. 27, 2010

(51) Int. Cl. A44C 5/00; A61B 5/117

(52) U.S. Cl. 40/633

(57) ABSTRACT

The present invention relates to identification systems for identifying a person or object, and more particularly to a winged identification tag for infants and small children. In one embodiment, an identification tag includes an identification portion having first and second opposite sides and first and second opposite ends, a strap portion extending from the first end of the identification portion, a wing extending from the first side of the identification portion, a curved notch forming curved corners where the wing meets the identification portion, and a removable cover piece underlying at least a portion of the wing.

### FIELD OF THE INVENTION

The present invention relates to identification systems for identifying a person or object, and more particularly to a winged identification tag for infants and small children.

### BACKGROUND OF THE INVENTION

Identification systems are used in many fields to provide a convenient and effective way to identify important information about a person, animal, or object. Identification systems often consist of a wristband or bracelet that is attached to a person around the wrist (or ankle, or other location) and that states information about the wearer. For example, these wristbands may be used to identify a patient in a hospital and to provide important information about the patient, such as the patient's name, the doctor's name, the patient's blood type, the patient's allergies, and other useful information. Wristbands may also be used to identify people admitted to a sporting event, concert, amusement park, or other similar establishments and events. Similar identification systems may be useful in many other situations where certain information about particular individuals, animals, or objects needs to be readily available. As used herein, the term “wrist” or “wristband” is not intended to limit the application of the identification band to a person's wrist, as it may be used at other locations on a person (such as an ankle) or on other objects.

Identification bands such as wristbands and bracelets are often made of a flexible material that is formed into a loop and then secured with a fastener. The fastener may be an adhesive or mechanical closure, and it may be attached to the wristband or it may be a separate element that is used to form the wristband into a loop.

The wristband may be pre-printed with information about the wearer, or it may have a space or area where information can be written by hand, printed, or attached by a label. For example, in the medical field, an adhesive label may be printed with patient information and then attached to the wristband. As long as the wristband remains attached to the patient, attending nurses, doctors, and other caregivers can easily locate this information before administering any treatment to the patient.

Wristbands are often used to identify an infant or young child in a hospital or other medical center. Infants and small children have small wrists, and as a result the wristband has a high degree of curvature as it wraps around the child's wrist. Barcodes printed on these wristbands can be difficult or impossible to scan with a barcode scanner due to the curvature of the band. Additionally, the band has a limited amount of space since the patient's wrist is so small, so it is difficult to fit all the necessary information on the wristband in a manner that is easily readable. Also, the information printed on the wristband may not be visible if the infant is lying down and covering a portion of the band.

Thus, there is still a need for an identification system that provides effective, safe, and convenient identification for infants and small children with small wrists.

### SUMMARY OF THE INVENTION

The present invention relates to identification systems for identifying a person or object, and more particularly to a winged identification tag for infants and small children. In one embodiment, an identification tag includes an identification portion having first and second opposite sides and first and second opposite ends, a strap portion extending from the first end of the identification portion, a wing extending from the first side of the identification portion, a curved notch forming curved corners where the wing meets the identification portion, and a removable cover piece underlying at least a portion of the wing.

In another embodiment, an identification system includes a direct thermal tape comprising a top layer, a bottom layer, and an adhesive between the top and bottom layers. The tape includes one or more cuts outlining an identification tag having an identification portion, a strap portion, and a wing extending from the identification portion. The strap portion has a length less than about three inches. The top layer has a fold line between the wing and the identification portion, and the bottom layer has one or more cuts defining a removable cover piece underlying the wing.

In another embodiment, an identification system includes a sheet having a top layer, a bottom layer, a release coating on at least a portion of the bottom layer, and an adhesive between the top layer and the bottom layer. The sheet is sized to pass through a printer. The system also includes an identification tag formed in the sheet and having an identification portion, a strap portion, and first and second wings on opposite sides of the identification portion. The system also includes a plurality of labels carried by the sheet. The sheet has a first portion wherein the top layer comprises paper and a second portion wherein the top layer comprises polyester. The tag is formed in the second portion.

In another embodiment, an identification system includes a wristband and a tag that has an identification portion carrying identification information, a strap portion extending from an end of the identification portion, and first and second wings extending from first and second opposite sides of the identification portion. The strap portion of the tag is looped around the wristband and behind the identification portion, and the first and second wings are folded over and secured to the identification portion with the strap portion secured between the folded wings and the identification portion.

In another embodiment, a method of identifying patient information in a medical setting includes providing a tag on a sheet or tape, the tag having an identification portion, a strap portion extending from an end of the identification portion, and a wing extending from a side of the identification portion. The method also includes printing information on the identification portion, separating the tag from the sheet or tape, and securing the tag to a patient wristband in a loop. In one embodiment, the tag is provided on a sheet having a plurality of polyester labels, and the method includes printing information on one of the polyester labels and securing the printed polyester label to a refrigerated item.

### BRIEF DESCRIPTION OF THE DRAWINGS

These and other features and advantages of the present invention will become appreciated as the same become better understood with reference to the specification, claims and appended drawings.

FIG. 1 is a top view of an identification tag according to an embodiment of the invention.

FIG. 2 is a top view of an identification tag with surrounding matrix removed according to an embodiment of the invention.

FIG. 3 is a top view of the identification tag of FIG. 2 attached to a wristband.

FIG. 4 is a back view of the identification tag of FIG. 2 attached to a wristband.

FIG. 5 is a top view of a series of identification tags according to an embodiment of the invention.

FIG. 6 is a top view of a sheet including two identification tags and labels, according to an embodiment of the invention.

FIG. 7A is a top view of a portion of a web of identification tags, according to an embodiment of the invention.

FIG. 7B is a perspective view of a roll of identification tags taken from the web of FIG. 7A.

FIG. 7C is a top view of an identification tag on a tape taken from the web of FIG. 7A.

FIG. 7D is a bottom view of an identification tag on a tape taken from the web of FIG. 7A.

FIG. 7E is a top view of a removable cover piece for the tag of FIG. 7D.

FIG. 7F is a bottom view of the tag of FIG. 7D removed from the tape, with the cover piece of FIG. 7E removed.

FIG. 7G is an enlarged view of the tag of FIG. 7A.

FIG. 7H is a partial front view of a tag attached to a wristband according to an embodiment of the invention.

FIG. 7I is a partial rear view of a tag attached to a wristband according to an embodiment of the invention.

FIG. 8 is a top view of a sheet including an identification tag and labels, according to an embodiment of the invention.

FIG. 9 is a top view of a sheet including an identification tag and labels, according to an embodiment of the invention.

FIG. 10 is a top view of a sheet including two identification tags and labels, according to an embodiment of the invention.

FIG. 11 is a top view of an identification tag according to an embodiment of the invention.

### DETAILED DESCRIPTION

The detailed description set forth below and the appended drawings are intended as a description of the presently preferred embodiments of an identification system provided in accordance with the present invention and are not intended to represent the only forms in which the present invention may be constructed or utilized. The description sets forth the features of the present invention in connection with the illustrated embodiments. It is to be understood, however, that the same or equivalent functions and structures may be accomplished by different embodiments that are also intended to be encompassed within the spirit and scope of the invention. As denoted elsewhere herein, like element numbers are intended to indicate like elements or features.

The present invention relates to identification systems for identifying a person or object, and more particularly to a winged identification tag for infants and small children. In one embodiment, identification tags are provided end-to-end in a tape or roll, with each individual tag separable from the other tags on the roll. The tag can be passed through a printer to print identification information on the tag, and after printing, the tag is removed from the surrounding matrix or tape. The tag is folded around a wristband for an object or person, such as an infant or small child in a hospital or other medical setting. The tail portion of the tag is looped around the wristband, and a cover piece is removed from the back surface of the identification area to expose adhesive underneath. The tail is adhered to this adhesive to secure the tag into a loop around the wristband. One or more foldable wings are then folded over and adhered over the tail to further secure the tag in place. The tag provides an extended surface for printing and reading identification information, and the tag can remain substantially flat and straight even when the wristband is curved about a small wrist. The tag can slide along the wristband so that it can be easily accessed and viewed even when the patient is lying down on the wristband. The tag can be slid onto a new wristband if the patient's wristband is replaced.

While the tag is described as being looped around a patient's wristband, it should be noted that the tag may be looped around other items as well, to provide information about the patient. For example, the tag may be printed with information and then looped and secured around a patient's tubing (such an IV tube), equipment near the patient (such as various vital sign monitors), the patient's bed (such as a baby's bassinet), or even the patient's clothing (such as around a loop or drawstring provided in a patient's clothing). The tag can be looped and secured around any suitable item to assist in identifying the patient. Additionally, this tag can be used in many settings, including non-medical settings such as sporting venues, concerts, and amusement parks. In the medical setting, it can be used for adults as well as babies and children. It is particularly useful for infants and small children with small wrists, but it is not limited to that usage.

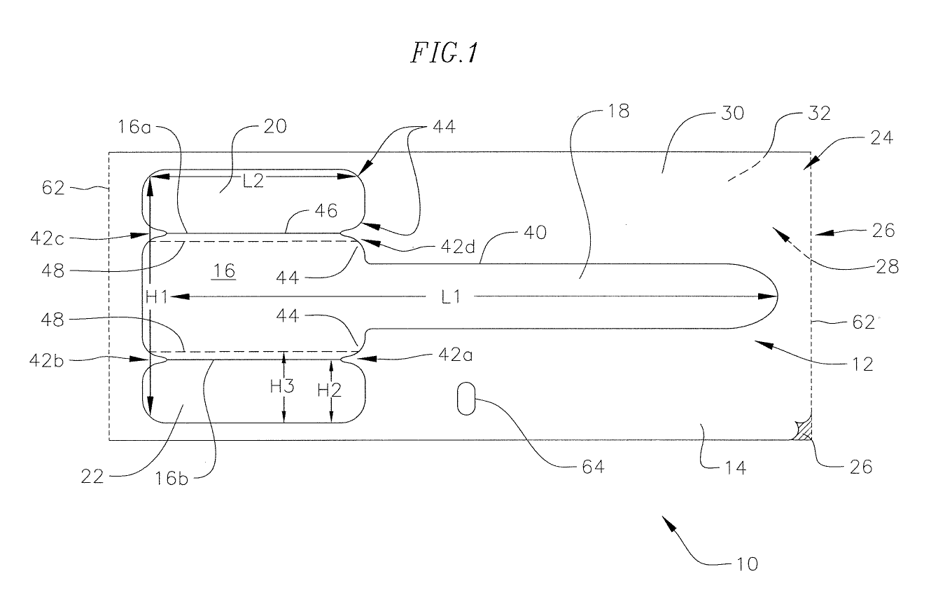

An embodiment of the identification tag system 10 is shown in FIG. 1. The system 10 includes a tag 12 situated on a tape or matrix 14. The tag 12 includes an identification portion 16, tail or strap portion 18, and two wings or ears 20, 22. The tail portion 18 extends from one end of the identification portion 16. Identification information can be printed on the identification portion 16 as well as on the wings 20, 22 and even on the strap portion 18. The tape 14 with the tag 12 can be passed through a thermal or other printer to print information on the tag. Alternatively, the information can be written or stamped onto the tag, or a label or sticker with information can be adhered to the tag. The identification portion 16 is shown as generally rectangular in shape, with curved corners, but it can be formed in other shapes and sizes according to the application or venue.

The tape or matrix 14 includes three layers: a top layer 24, a bottom layer 28, and an adhesive layer 26 between the top and bottom layers. In one embodiment, all three layers are coextensive with each other. In one embodiment, the bottom layer 28 includes a release coating facing the adhesive layer. (This release coating may also be referred to as a fourth layer of the matrix.) In such an embodiment, the adhesive is a relatively permanent adhesive, and the release coating enables the bottom layer to be removed from the adhesive. As a result, portions of the bottom layer can be removed to expose the adhesive, as described in further detail below. The adhesive remains adhered to the top layer 24.

The top layer 24 is a label stock, paper layer, polyester or other synthetic material or fabric, or any material suitable for receiving printed information. For example, when the identification tag system 10 is designed to pass through a laser printer, the top layer 24 is a suitable paper or label stock. When the identification tag system is designed to pass through a thermal printer, the top layer 24 is a thermal paper that changes color in response to heating, such that identification information can be printed on the paper. The bottom layer 28 is a release liner or any suitable label stock or paper layer. Both the top and bottom layers should be suitable for contact against a patient's skin without causing irritation.

The release coating on the bottom layer 28 can be any suitable coating that enables the adhesive to release from the bottom layer, rather than permanently adhering. In one embodiment, the release coating is a silicone coating. As described in further detail below, the release coating may be present in some areas of the matrix or tag and absent in other areas, or it may be absent altogether.

In one embodiment, the adhesive layer 26 is uniform throughout the area of the tag, meaning that the same adhesive or mix of adhesives is used throughout the tag 12 and matrix 14. Use of a single adhesive or mix of adhesives simplifies the manufacturing process and reduces cost. However, in other embodiments, different adhesives are provided at different portions of the tag. For example, a removable adhesive can be provided under the identification portion and wings, so that the bottom layer 32 can be removed to expose the adhesive there, while a more permanent adhesive can be provided over the strap portion 18. Other arrangements of adhesives can be used as well. In such a case, the release coating may be omitted, as the adhesive is chosen to either release or bond to the layers, as desired.

The three layers 24, 26, 28 are adhered together to form one coextensive tape or matrix 14, with a top surface 30 and back surface 32.

One or more wings 20, 22 extend from the identification portion 16. The identification portion 16 includes a first side 16a and a second side 16b opposite the first side. The first wing 20 extends from the first 16a, and the second wing 22 extends from the second 16b. The identification portion also has first and second opposite ends, with the strap extending from the first end. In this embodiment, two wings are provided, but in other embodiments a single wing may be provided, extending from the first or second side of the identification portion or even from the second end of the identification portion, opposite the strap.

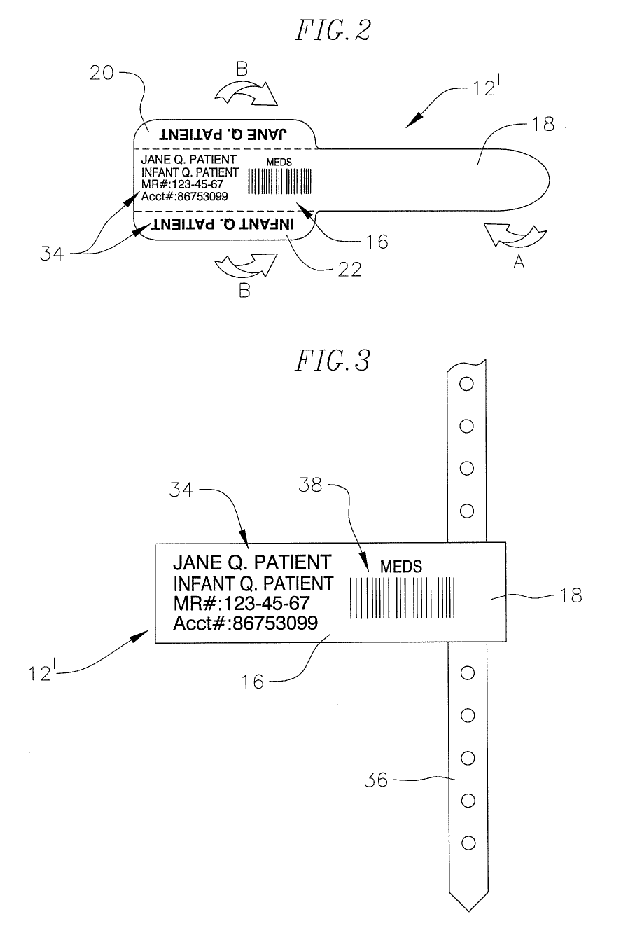

The wings 20, 22 are designed to fold toward the back surface 32 of the tag 12 when the tag 12 is removed from the tape 14 and put into use. The wings fold along fold lines 46, which are dents or creases in the top layer 24 of the tag. This folding action is shown in FIGS. 2-4. FIG. 2 shows a tag 12′ according to an embodiment of the invention. This tag 12′ has been printed, stamped, labeled, or otherwise provided with identification information 34 and has been removed from a sheet or tape. The tag 12′ is ready to be attached to a wristband to provide the identification information 34 about the wearer. As indicated in FIG. 2, the strap portion 18 is folded down in the direction of arrow A, toward the back surface 32, and looped around the wristband 36. The release liner 28 is removed from the identification portion 16, and the strap portion 18 is then adhered to the exposed adhesive 26 on the back surface 32 of the identification portion 16. The wings 20, 22 are then folded down toward the back surface 32 in the direction of arrows B. The release liner is removed from the wings 20, 22, which are then adhered over the strap portion 18 to further secure the tag into a loop about the wristband 36.

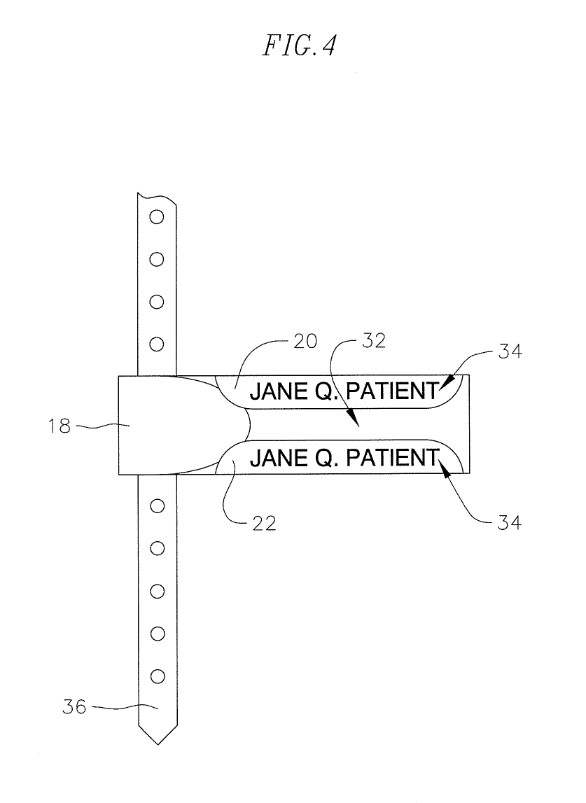

The securely looped tag 12′ is shown in FIGS. 3 and 4. The strap 18 has been looped around the wristband 36 and secured to the back surface of the identification portion 16. The wings 20, 22 have been folded down over the strap. In this configuration, the identification information 34 is visible on the front surface of the identification portion 16, as shown in FIG. 3. A nurse, doctor, other medical caregiver, or other attendants in other settings can view the top surface of the tag to view the identification information 34. The tag 12′ extends laterally or perpendicularly out from the wristband 36 and does not curve (or curves only slightly) with the wristband around the patient's wrist. This enables the tag 12′ to remain substantially flat and easy to read. Barcode 38 is more easily and reliably scanned when it is printed in this manner on the perpendicular tag than when it is printed along the circumference of the curved wristband 36. In the embodiment of FIG. 3, the tag 12′ has a rectangular shape once it has been looped and secured around the wristband 36.

A rear view of the tag 12′ in use is shown in FIG. 4. In this view, the end of the strap portion 18 is visible, having been looped around the wristband 36 and adhered to the back surface 32 of the identification portion 16. The wings 20, 22 are also visible, having been folded over on top of the strap 18 to further secure it in place. The one or more wings 20, 22 may also have identification information 34 printed or otherwise provided on them. A caregiver or attendant can lift the tag 12′ and view the information 34 provided on these two wings 20, 22. The wings thus provide an additional area for information to be printed and displayed.

Moreover, the wings enable information to be displayed on the back surface of the tag with only one pass through a printer. The identification tag system, such as the system 10 shown in FIG. 1, can be passed through a printer just once, and information can be printed on the identification portion 16 and wings 20, 22 in the same pass through the printer. The system does not have to be turned over to expose the back surface and passed through the printer a second time, in order to display information on both the front surface 30 and the back surface 32. Because the wings initially face up, as shown in FIG. 1, but are then folded over, as shown in FIG. 2-4, they enable the tag to display information on both the top and bottom surfaces once assembled. In the embodiment shown in FIGS. 2 and 4, the tag 12′ can be used in a hospital to identify a mother and baby. One wing 20 is printed with the mother's name or other identifying information, and the other wing 22 is printed with the baby's name or identifying information, so that the tag when attached to the baby's wristband identifies the baby as well as the mother. This is just one example of how the printed wings can be used. The wings can also be printed such that when folded, the information on both wings is readable from one direction (as shown in FIG. 4), or if desired, they can be printed with the text reading in opposite directions.

The tag 12′ can slide freely along the wristband 36 because the adhesive 26 between the top and bottom layers 24, 28 is not exposed along the strap portion 18. The bottom layer 28 remains attached to the top layer 24 along the strap portion 18, covering the adhesive 26 between the bottom and top layers. Thus, when the strap portion 18 is looped around the wristband, it will not stick or adhere to the wristband. This enables the tag to freely slide around the wristband so that it is easier to read, or moved to a more comfortable position for the patient.

Referring back to FIG. 1, the tag 12 is outlined in the tape 14 by an outer tear line or perforation 40. This tear line 40 outlines the tag 12 and makes it easy for the user to remove the tag from the tape 14. This tear line 40 includes notches 42a, 42b, 42c, 42d at the four corners of the identification portion 16, where it meets the two wings 20, 22. The notches 42 have a curved, concave contour, creating rounded corners 44 on both the identification portion 16 and the wings 20, 22. Thus, the four corners of each wing 20, 22 and of the identification portion 16 are all rounded. These rounded corners provide additional comfort to the patient when the patient rubs or lays on the tag 12. When the tag 12 is looped around the wristband and the wings 20, 22 are folded down, there are no sharp edges or corners that could cut into an infant or child wearing the wristband and tag.

In one embodiment, the tag 12 is approximately 5 inches in length L1 and 2 inches in height H1. The identification portion and wings are approximately 1¾ inches in length L2, and the wings are approximately ½ inch in height H2. The length of the tape 14 between tear lines 62 is approximately 5½ inches. These dimensions are just one example, and other dimensions can be used to create tags of various sizes and shapes. In one embodiment the tag is no more than about 5 inches in length.

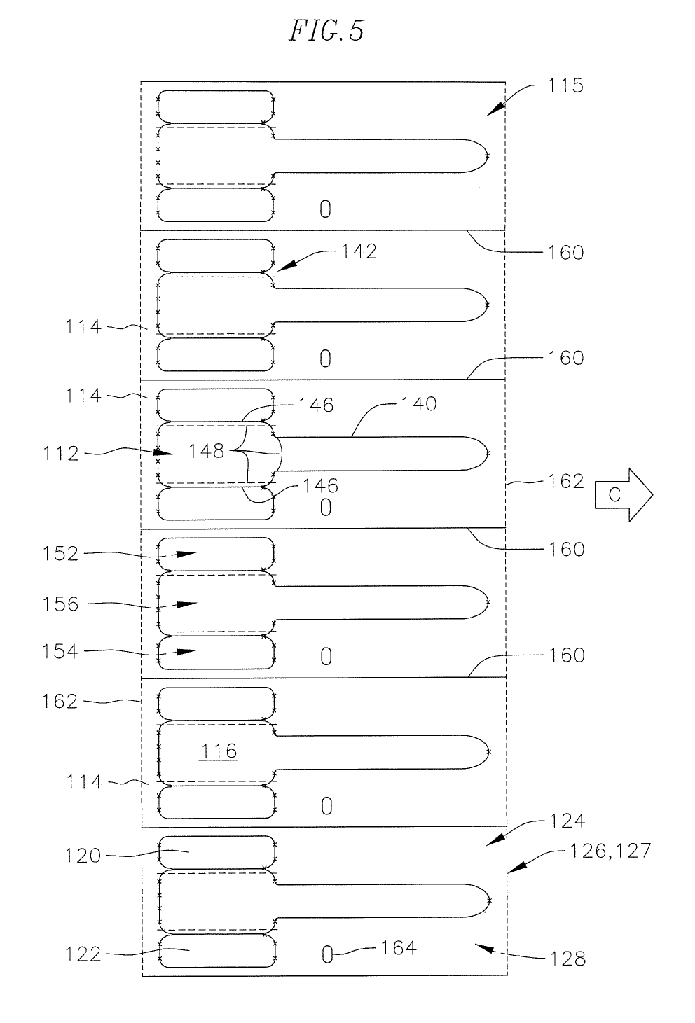

FIG. 5 shows a series of tags 112 as they are arranged during the manufacturing process according to one embodiment of the invention. FIG. 5 shows just one column of tags 112, but these columns repeat so that the tags are arranged in rows as well. Each row includes tags aligned end-to-end, and each column includes tags arranged side-by-side. The terms “column” and “row” are illustrative terms used for reference in FIG. 5 and are not meant to be limiting.

The tags 112 are provided on a web 115 that is eventually separated into several tapes 114 of end-to-end tags 112 (in this embodiment, six tapes 114). The web 115 is passed through a die cutting machine to create the tags 112 and separate the tapes 114. The web includes four layers: bottom layer 128, release coating 127 (disposed on the bottom layer), adhesive layer 126, and top layer 124. The web is passed into the die cutting machine, which cuts outer tear lines 140 through the top layer 124 of the web 115, creating an outline of the tag 112. This outline includes notches 142. The outline 140 is formed by cutting some portions of the outline all the way through the top layer and bottom layer of the band, and cutting other portions of the outline through the top layer only (or through the top layer and partially through the bottom layer). This makes it easy for the user to remove the tag from the tape 114 for use, while leaving the tag attached to the tape before it is ready for use.

Fold lines 146 are created by denting or creasing the top layer 124, rather than cutting through the top layer. The dented fold lines 146 make it easier for the user to fold the wings 120, 122 along a straight line, and it indicates to the user where the wings should be folded.

Tear lines 148 are cut into the bottom layer 128 to create three separate removable cover pieces 152, 154, 156 in the bottom layer 128. In one embodiment, the tear lines 148 are cut through the bottom layer 128 but do not extend into the top layer 124. The first and second cover pieces 152, 154 underlie the first and second wings, respectively, and the third cover piece 156 underlies the identification portion. In use, the third cover piece 156 is removed first, exposing the adhesive under the identification portion 116. The strap 118 of the band is folded around the patient's wristband and adhered to this exposed adhesive, where cover piece 156 was removed. The cover pieces 152, 154 are then removed and the wings 120, 122 are folded over to sandwich the strap and secure the tag in a loop about the wristband. In another embodiment, only two cover pieces 152, 154 are provided, and the area under the identification portion is not uncovered to expose the adhesive. Instead, the strap is folded over to form the tag into a loop, and the cover pieces 152, 154 are removed to fold the wings 120, 122 over and adhered the strap in place.

In one embodiment, the top and bottom tear lines 148 are offset from the two fold lines 146. As shown in FIG. 1, the fold lines 46 are located a distance H2 from the outer edge of the wing, and the tear lines 48 are located a larger distance H3 from the outer edge of the wing. When these two lines are created at the same place, with the fold lines 46 in the top layer and the tear lines 48 in the bottom layer, the tag can become weak at that location and can rip or tear. Offsetting the fold lines and tear lines improves the durability of the tag.

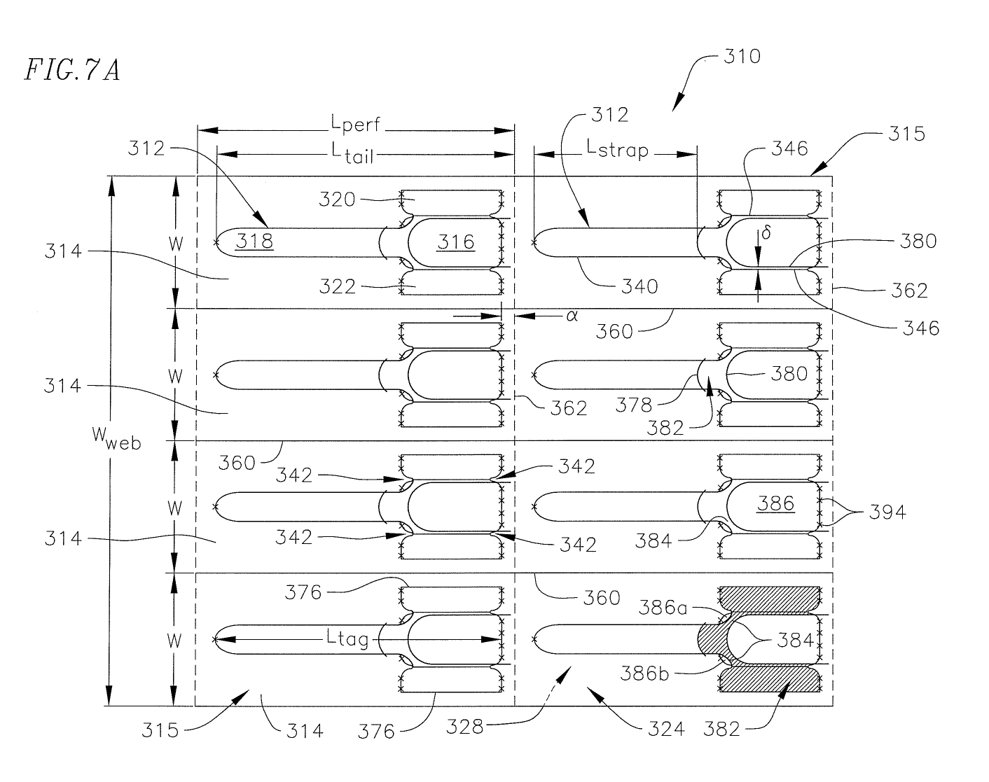

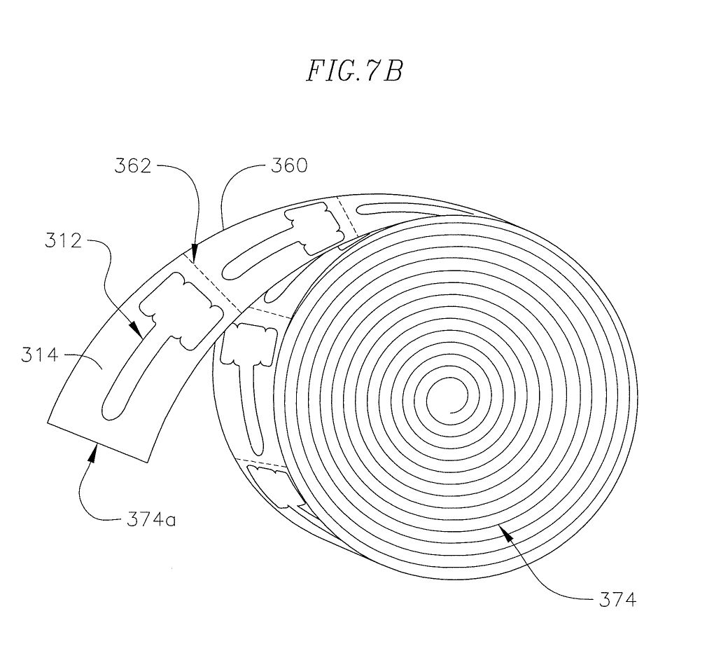

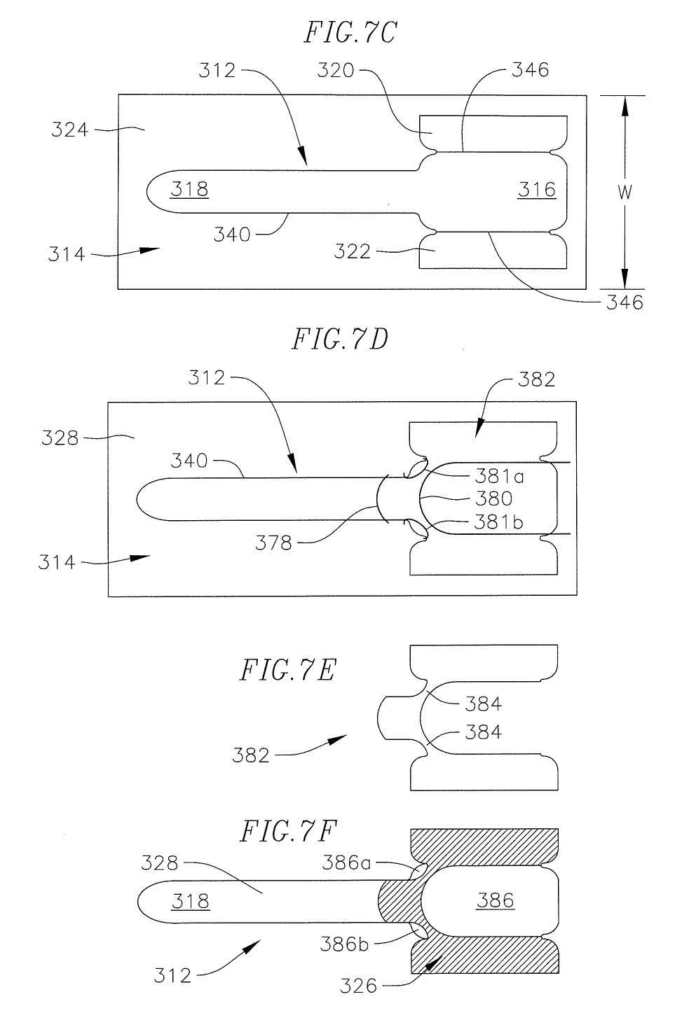

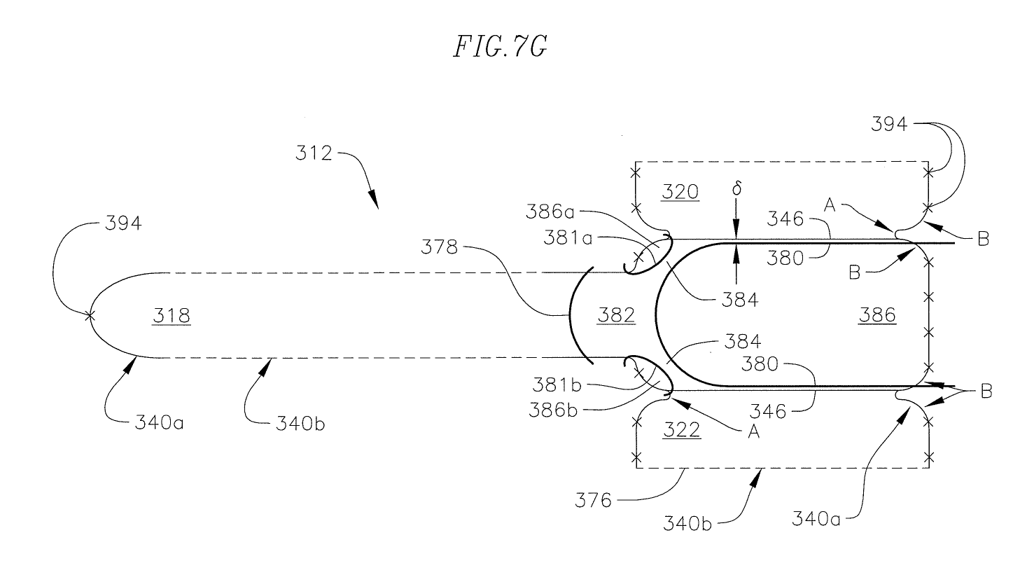

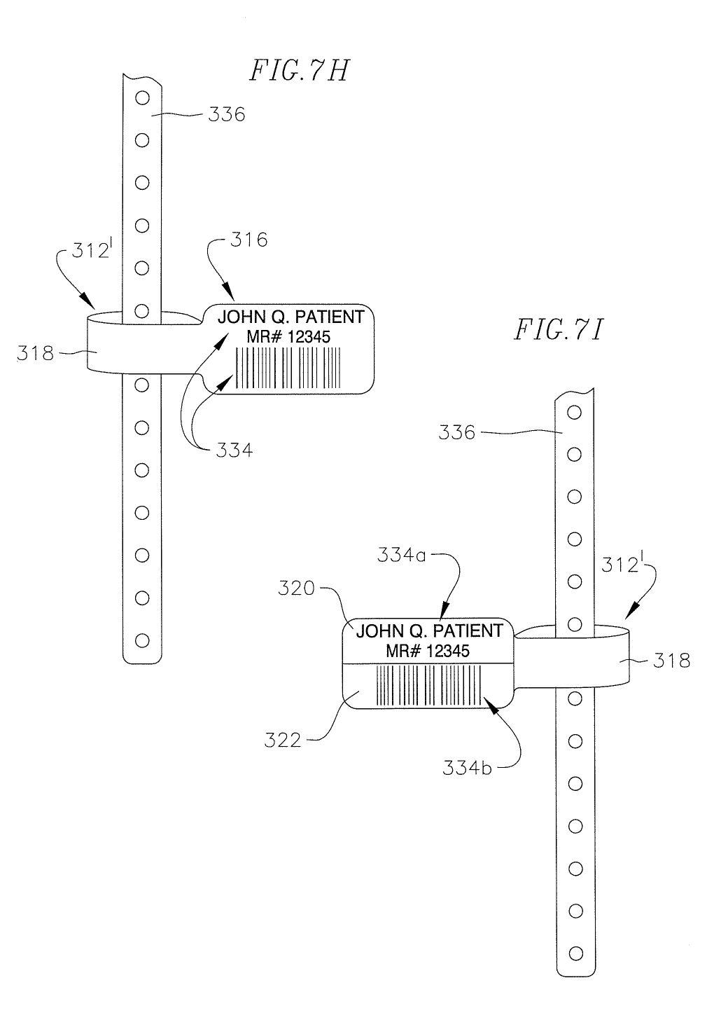

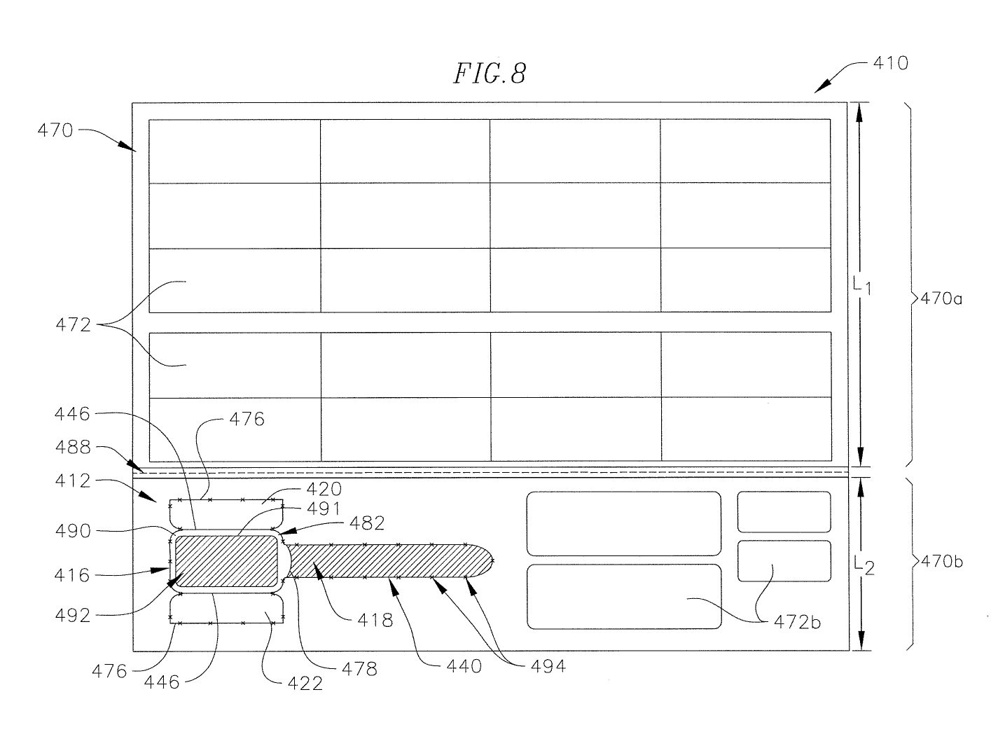

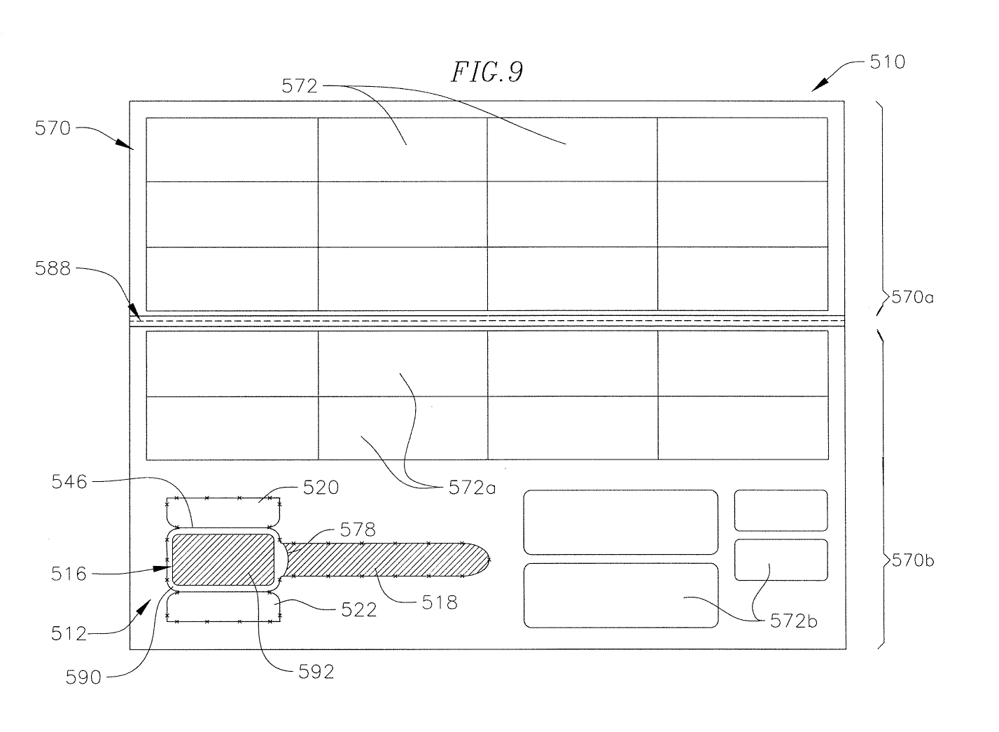

Referring again to FIG. 5, the cuts and impressions (outer tear line 140, fold lines 146, and bottom tear lines 148), the tag 112 is created in the web 115. As shown in FIG. 5, several tags 112 are created side-by-side along the web 115. As the web is passed through the die cutting machine, additional tags are created in repeating columns, creating six rows of end-to-end tags. Although six tags are shown in FIG. 5, more or fewer tags can be included, depending on the size of the web and die cutting machine and the desired number of tags and rolls. Once the tags are cut into shape as shown in FIG. 5, the die cutting machine forms tear lines 160, which separate the tags into tapes 114 of end-to-end tags. Each of these rows of tags is wound into a roll (see FIG. 7B, discussed below). This process creates tapes or rolls of tags 112 with the tags arranged end-to-end. Tear lines 162 are formed between adjacent end-to-end tags so that an individual tag can be removed from the end of the roll for use.

The tags 112 in tape form are designed for use with thermal printers. The tape can be fed through a thermal printer that is designed to accept a tape of that size. The printer prints identification information on the tag, and may use a punched-out oval 164 or a black mark or other visible indicator to signal to the printer when to begin printing. The printer may print on the identification portion and/or one or both wings. In this embodiment, the top layer 124 of the tags is a thermal paper that responds to heat from the thermal printer to create an image.

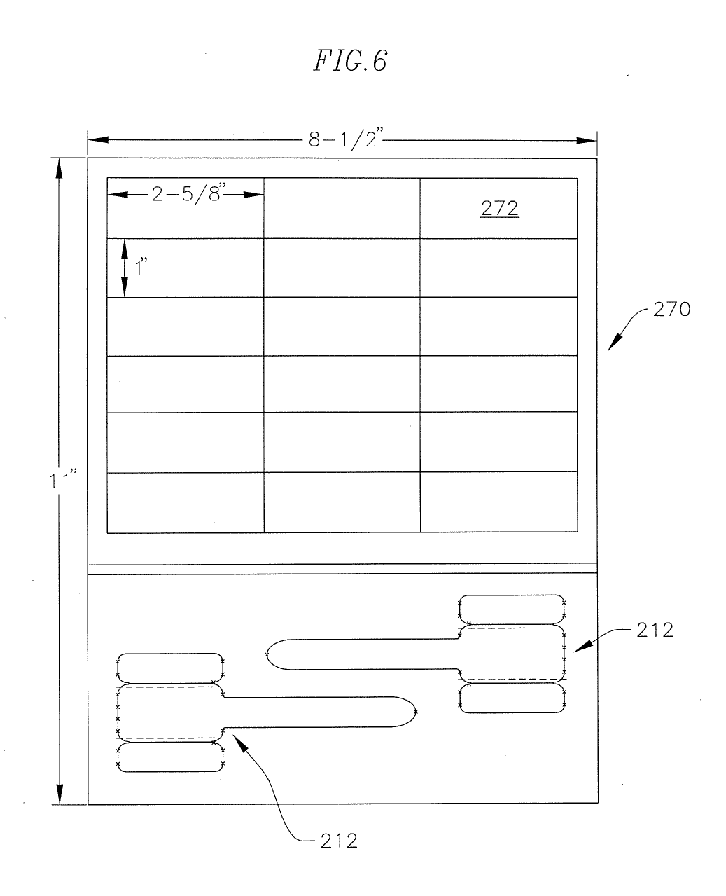

In another embodiment, shown in FIG. 6, tags 212 are designed for use with a laser or inkjet printer. The tags 212 in FIG. 6 are situated on a sheet 270 that is designed to pass through a standard laser or inkjet printer. In one embodiment, the sheet 270 is 8½ inches by 11 inches in size. The sheet 270 has two tags 212 near the bottom of the sheet and several rectangular labels 272 above the tags. When this sheet is passed through the printer, the printer can print identification information on the tags as well as the labels. The tags can be attached to an infant or child wristband as described above, and the labels can be attached to medical charts, medications, bills, and other items to be associated with the patient. In one embodiment, the first tag on the sheet is printed with a baby's identification information and is attached to the baby's wristband, and the second tag is printed with a mother's identification information and is attached to the mother's wristband. In another embodiment, one tag is attached to a baby's wristband and the other tag is attached to the baby's ankle-band. The exact configuration of tags and labels shown in FIG. 6 is just one example, and the sheet 270 can include other arrangements of tags, labels, cards, and other printable items. Additionally, in another embodiment, the sheet 270 is a sheetlet including only the bottom portion of the sheet with the tags 212, and no labels.

An identification system 310 according to another embodiment of the invention is shown in FIGS. 7A-7G. FIG. 7A shows a web 315 after it has passed through a die cutting machine (or similar device) to form tags 312 end-to-end on separable tapes 314. The tapes 314 are separated along tear lines 360 to form individual tapes of width W, which can be wound or rolled into rolls 374, as shown in FIG. 7B. A tag 312 on an individual tape 314 is also shown in FIGS. 7C and 7D, and an enlarged tag is shown in FIG. 7G. The tags 312 have a strap portion 318, an identification portion 316, and one or more wings 320 and 322. The wings 320 and 322 connect to the identification portion 316 at curved notches 342, which create curved corners when the wings are folded over.

In one embodiment, the outer edges 376 of the wings are flat or straight, without rounded corners. The wings are folded over onto the back surface of the identification portion 316, so the flat edges 376 are positioned in the middle of the identification portion on the back surface of the tag. The flat edges, without any curved cutouts or notches, provide greater coverage over the back surface of the tag, covering the exposed adhesive without causing discomfort to the patient.

The tapes 314 are cut in several places to create the tags 312, as shown in FIGS. 7A, 7C, 7D, and 7G. FIGS. 7A and 7G show all of the cuts and lines in one view, although some are made in the top layer 324 of the tape and others are made in the bottom layer 328. FIGS. 7A and 7G shows all of the cuts together, to more clearly show the operation of the die cutting machine to create the tags. For clarity, FIGS. 7C-7F are also provided to show the separate cuts made in the top and bottom layers. FIG. 7C shows a top view of the tag, showing the lines and cuts in the top layer 324. FIG. 7D shows a bottom view of the tag, showing the lines and cuts in the bottom layer 328. FIG. 7E shows the shape of the removable cover piece 382, and FIG. 7F shows a bottom view of the tag after the cover piece has been removed.

As shown in FIGS. 7A, 7C, 7D, and 7G an outline 340 creating the perimeter of each tag 312 is formed by cutting through both the top layer 324 and the bottom layer 328 of the tape 314, except at retention points 394 (noted by x's) where a cut is not made. The tag 312 is then easily removed from the tape 314, yet stays in place due to the uncut points 394 that retain the tag 312 to the tape 314 until it is ready for use. These retention points 394 are omitted from FIGS. 7C and 7D for clarity.

As shown in FIG. 7G, in one embodiment the outline 340 includes portions 340a and 340b. The portion 340a is a cut that is made all the way through the tag, through both the top and bottom layers. The cut extends completely through the matrix material 315 at the portions 340a. Thus the tag 312 is completely separated from the tape 314 at these portions 340a. The portions 340b, indicated by dotted lines, are a cut with a different depth. Instead of passing all the way through the matrix, the portions 340b pass partially through the material. In one embodiment, the portions 340b are cut all the way through the top layer 324 and partially through the bottom layer 328. As a result, the tag 312 remains partially attached to the tape 314 at these portions 340b. The partial cut enables the tag 312 to be easily pulled away from the tape 314 at these portions 340b. Thus the tag 312 remains with the tape 314 prior to use, but can be easily separated.

FIG. 7G shows one example of an arrangement of portions 340a, 340b with cuts of different depths. In this embodiment, the through cut portions 340a include the tip of the strap 318, the opposite end of the strap, and the notches 342. The partially cut portions 340b include the middle of the strap, the straight edges 376, and the free end of the identification portion. It is noted that the portion 340b is identified by dotted lines in order to distinguish it in the figure from portion 340a, and not to indicate that the cut 340b is intermittent. Rather, the partial cut in portions 340b extends continuously through those portions.

As shown in FIGS. 7A, 7C, and 7G, fold lines 346 are created by denting the top layer 324 where the wings 320, 322 are folded over. Each tag has two fold lines, one between each wing and the identification portion. The fold lines 346 are positioned such that when the two wings 320, 322 fold over, their flat edges 376 meet in the center of the identification portion 316. In one embodiment, the wings meet without overlapping. As a result, printed information on both wings is visible without being covered by the other wing.

As shown in FIGS. 7A, 7D, and 7G, a total of four cuts are made in the bottom layer 328 of the tape to form the removable cover piece 382 that underlies the wings 320, 322. The user removes this cover piece 382 to expose the adhesive 326 between the top and bottom layers. The cover piece 382 is shaded in the tag 312 at the bottom right of FIG. 7A, to more clearly show the shape of the cover piece. The cover piece 382 is also shown in FIG. 7E, and the exposed adhesive 326 is shown in FIG. 7F.

The four cuts made in the bottom layer 328 to form the removable cover piece are shown in FIG. 7D and in FIG. 7G (shown in bold lines for clarity). The four cuts are identified by numbers 378, 380, 381a, and 381b. In one embodiment, the cuts pass all the way through the bottom layer 328, and do not pass into the top layer 324.

The first cut 378 is made through the bottom layer 328 across the strap portion 318, near the identification portion 316. In the embodiment shown, this cut 378 is curved, convex toward the tail end of the strap 318. This convex curve helps the user grasp the cover piece 382 to remove it. In other embodiments, the cut 378 can have more or less or different curvature, or can be straight.

A second cut 380 through the bottom layer 328 divides the identification portion 316 into two portions: a first removable portion 384 that is part of the removable cover piece 382, and a second reinforcing portion 386 that is not removed. These portions are shown in FIGS. 7D-7G. The second reinforcing portion 386 remains adhered to the top layer 324 of the tag 312, even after the user removes the cover piece 382. This reinforcing portion 386 reinforces the identification portion 316, retaining the thickness of both the top layer and bottom layers. The information printed on the top side of the identification portion is supported by the thickness of both the top and bottom layers, where the reinforcing portion 386 is located, so that is it not as easily wrinkled, bent, or torn.

In the embodiment shown, the cut 380 forming the first and second portions 384 and 386 is U-shaped. The curved portion of the cut 380 passes through the identification portion, near the strap, and then the straight portion of the cut continues parallel to the fold lines 346 next to each wing. Each fold line 346 is offset from the cut 380 by a small distance δ (as shown in FIGS. 7A and 7G) so that the tag 312 is not unduly weakened at this point by overlapping the fold line 346 (in the top surface 324) with the cut 380 (in the bottom surface 328). In one embodiment, δ is about 0.015 inches.

In the embodiment shown, the cut 380 is positioned such that the reinforcing portion 386 occupies a majority of the area of the identification portion 316 (see FIG. 7F).

In one embodiment, two additional cuts 381a and 381b are formed in the bottom layer 328, as shown in FIGS. 7D and 7G. These cuts extend across the corners of the identification portion 316 nearest the strap 318. These cuts create corner portions 386a and 386b, which are portions of the bottom layer that remain with the identification portion even after the cover piece is removed. These corner portions 386a, 386b prevent any adhesive 326 from remaining exposed at these corners after the cover piece is removed and the wings folded over. Given the placement of the fold lines 346 and the shape of the wings 320, 322, with their rounded corners, it is possible that when the wings are folded over, they will not be aligned exactly to cover the corners at 386a, 386b. If this happens, then the adhesive 326 could be exposed at these portions, and the tag could then stick to the patient, wristband, or other items. Accordingly, in one embodiment, the cuts 381a, 381b are provided so that the corner portions 386a, 386b remain adhered to the tag, covering the adhesive at those corners. As a result, less adhesive is exposed when the cover piece is removed, and the wings 320, 322 do not have to be lined up exactly along the notches in order to cover all of the exposed adhesive.

Between the main portion 386 and the corner portions 386a, 386b is the bridge portion 384 of the cover piece 382. The bridge portion 384 is a narrow portion that extends between each wing and the strap portion 318 (see FIGS. 7A and 7E), connecting the removable cover piece between the strap portion and the wings, so that the cover piece 382 is all one continuous piece.

The cuts 378, 380, 381a, 381b create a removable cover piece 382 with the shape highlighted at the bottom right of FIG. 7A and shown in FIG. 7E. This cover piece 382 is removable in one piece to expose both wings 320, 322. Thus, the user can expose the adhesive 326 on the bottom surface of both wings 320, 322 in a single motion, by lifting the removable cover piece at the curved cut 378 and peeling it away from the tag 312. When the cover piece is removed, the adhesive 326 between the bottom and top layers is exposed (see FIG. 7F). When the strap 318 is looped around the patient's wristband and the wings 320, 322 are folded over, the strap and wings will cover this exposed adhesive so that no adhesive remains exposed after the tag is secured.

In other embodiments, the removable cover piece 382 can take on other shapes and sizes, depending on the area of the identification portion 316 to be reinforced, the amount of exposed adhesive desired, and other factors. The cover piece can be one piece, as shown in FIG. 7E, or can be split into two or more pieces that are removed individually and that may or may not touch each other.

The particular layout of the tags 312 on the matrix 315 in FIG. 7A is just one example of how the tags can be arranged. Other arrangements may be used. In the arrangement shown, the tag is spaced by distance α from the tear lines 362. In one embodiment, α is 0.25 inches. The width of the tape 314 and the distance between tear lines 360 is W, which in one embodiment is 2.25 inches. The width of the web Wweb is W multiplied by the number of tapes 314 included, which is four in FIG. 7A. Thus Wweb is 9 inches in one embodiment. The distance between perforations 362 is shown as Lperf, which is 5.5 inches in one embodiment. Also, the distance from one perforation 362 to the tail end of the strap 318 is labeled as Ltail, which in one embodiment is 5.25 inches. The length of the tag 312 itself, Ltag, is 5 inches in one embodiment, and the length of the strap Lstrap (from the end to the cut 378) is 2.85 inches in one embodiment. In one embodiment, the length of the strap is less than the circumference of a baby or small child's wrist, such as less than about 3 inches, or in one embodiment less than about 2 inches. In one embodiment, the length of the strap is about 2 inches and in another embodiment about 3 inches.

The curvatures of the notches and the corners of the wings and identification portion are identified by the letters A and B in FIG. 7G. The radius of curvature at the notches (letter A) is 0.0468 inches, in one embodiment. In other embodiments it is about 0.05 inches or less than about 0.05 inches. The radius of curvature of the rounded corners of the wings and identification portion (shown by letter B) is 0.2187 inches, in one embodiment. In another embodiment it is about 0.2 inches.

FIG. 7B shows a roll 374 of tags 312 arranged end-to-end on a tape 314. This tape 314 has been separated along tear line 360 from adjacent tapes on the web 315 from FIG. 7A. The free end 374a of the roll can be fed into a thermal printer, so that the tags 312 can be printed with patient information and then removed from the roll by tear line 362. The tag can then be removed from the tape 314 along outline 340, and wrapped around a patient's wristband.

In one embodiment, the material of the matrix 315 is designed for thermal printing. The matrix includes the top layer 324 and bottom layer 328 with an adhesive layer 326 between the top and bottom layers. In one embodiment, the adhesive is a relatively permanent adhesive and is coextensive with the top and bottom layers. The top layer 324 is a direct thermal facestock, which could be paper, polypropylene, or another suitable paper or synthetic material that can be thermally printed. The bottom layer 328 is a release liner and includes a release coating, such as a paper with a synthetic silicone coating, facing the adhesive layer, so that the bottom layer can be released from the adhesive.

FIGS. 7H and 7I show top and rear views of a tag 312′ according to an embodiment of the invention, looped around a wristband 336. The top view of the tag in FIG. 7H shows patient identification information 334 printed on the top surface of the tag in the identification portion 316. The rear view of the tag in FIG. 7I shows the two wings 320, 322 folded over to secure the strap 318 in a loop around the wristband 336. In the embodiment shown, the first wing 320 has information 334a, and the second wing 322 has information 334b. The two wings align so that the information 334a, 334b is not blocked by either wing. Thus, patient information can be printed and visible on both sides of the tag 312′. Additionally, in the embodiment shown, the strap 318 provides some clearance around the wristband so that it can easily slide to a new location, for better visibility or more comfort. In other embodiments, the strap can be shorter, with less clearance around the wristband. The clearance can also be adjusted by moving the tail end of the, strap further along the back of the identification portion 316 before securing it with the wings, to form a smaller loop.

An identification system 410 according to another embodiment of the invention is shown in FIG. 8. The identification system 410 includes a sheet 470 including a tag 412 and a plurality of labels 472. The sheet 470 is divided into two portions: a first paper portion 470a and a second polyester or synthetic portion 470b. The second portion 470b may be referred to herein as the “polyester” portion of the sheet, although polyester is simply one embodiment. It should be understood that the second portion of the sheet is not limited to polyester, and embodiments include polyester as well as other synthetic materials such as various polymer, plastic, or fabric materials.

The portions 470a and 470b are divided by a perforation or tear line 488. The two portions can be pulled apart from each other at this perforation. Both portions of the sheet include a top layer, a bottom layer, a release coating, and an adhesive layer between the top and bottom layers. However, the top layer in the paper portion 470a is a paper or label stock, and the top layer in the polyester portion 470b is polyester or suitable synthetic material. The polyester is used in order to provide a more comfortable tag 412 that is more flexible and soft than paper tags. The polyester is also more durable than paper, in some applications, as it is more difficult to tear or rip. In one embodiment, both portions 470a, 470b have the same backing/bottom layer, such as a paper backing liner with a release coating.

The paper portion 470a includes paper labels 472. The polyester portion 470b also includes one or more labels 472b, which have polyester as the top layer. However, the labels are optional, and in another embodiment, a sheet 470 is provided with one or more tags 412 and no labels.

The tag 412 includes a strap portion 418, an identification portion 416, and wings 420 and 422 with fold lines 446. In the embodiment shown, the adhesive layer between the top and bottom layers of the tag is not uniform across the entire sheet 470. For most of the sheet, including both the paper portion 470a and the polyester portion 470b, the adhesive is coextensive with the top and bottom layers. However, in this embodiment, no adhesive is provided along the strap portion 418 of the tag 412. That is, the adhesive is absent between the top and bottom layers of the tag 412 along the strap portion 418. As a result, when the tag 412 is removed from the sheet, the polyester top layer of the tag separates from the backing layer along the strap portion 418. Once the tag 412 is removed from the sheet, the strap portion 418 is a single ply, carrying only the top polyester layer. The one-ply strap portion 418 is thinner and more flexible than a two-ply (or greater) strap, providing greater comfort for the patient. Also, it has no adhesive on it, so the tag 412 can slide freely along a patient's wristband once the tag has been looped and secured. The remainder of the tag 412 and the sheet 470 includes adhesive between the top and bottom layers.

For most of the sheet 470, a release coating is provided between the bottom layer and the adhesive layer, so that the tag 412 and labels 472, 472b can be removed from the bottom layer. However, the release coating between the bottom layer and the adhesive layer is absent at an area 492 in the center of the identification portion 416 of the tag 412. This area 492 may be referred to as a reinforcing area or bonded area, where the adhesive bonds to both the top layer and the bottom layer, keeping the two layers adhered together at this area. In the embodiment shown, the bonded area 492 is located inside a border 490 within the identification portion 416. The release coating is absent in the bonded area 492 but present elsewhere along the tag 412. The area 492 is shaded in FIG. 8 for clarity.

The bonded area 492 reinforces the identification portion 416, retaining the thickness of both the top layer and bottom layers. The information printed on the top side of the identification portion is supported by the thickness of both the top and bottom layers, where the bonded area 492 is located, so that is the identification area not as easily wrinkled, bent, or torn.

In the embodiment shown, the bonded area 492 is located at the center of the identification portion 416, inside of the border 490. These two areas 490, 492 can be arranged and shaped differently in other embodiments. Additionally, the border 490 can be entirely absent, such that the entire identification portion 416 is permanently bonded to the backing, or the bonded area 492 can be absent, such that the entire identification portion 416 is removably adhered. In the embodiment shown, the area 492 occupies a majority of the identification portion, with a relatively small periphery 490 surrounding it.

In an alternative embodiment, the sheet 470 does not include a release coating, and the adhesive layer is varied along the sheet 470 to provide either a bonded area or releasable area. For example, a relatively permanent adhesive is used at the bonded area 492 to retain the top and bottom layers together, and a removable adhesive is used along the rest of the tag 412, and the labels 472, 472b, so that they can be removed from the bottom layer. The term “permanent” or “relatively permanent” is used here as a relative term, to compare to a removable adhesive, and to indicate an adhesive where the top and bottom layers are not easily pulled apart. A release coating can be used to enable the layers to be pulled apart, with the permanent adhesive remaining with one layer and releasing from the release-coated layer.

As shown in FIG. 8, the bottom layer 428 is cut to form a removable cover piece 482 under the wings 420, 422 and border 490. A first convex curved cut 478 creates curved end for the user to grasp the cover piece 482 and remove it. A second cut 491 outlines the bonded area 492 so that the cover piece separates there, leaving the bottom layer attached at this area 492. These cuts give the cover piece 482 a donut shape, leaving the bonded area 492 behind when the cover piece 482 is removed. The cover piece 482 is all one piece and can be removed in one motion, thereby exposing the adhesive between the cover piece and the top polyester layer. The strap portion 418 and the wings 420, 422 are then looped, folded, and secured as described above.

In one embodiment, the tag 412 is about 5 inches in length, and about 1.875 inches between flat edges 476. The bonded area 492 is about 1.5 inches by 0.69 inches. The distance between the flat edge 476 and the fold line 446 is about 0.47 inches and the length of the wing is about 1.75 inches. The strap is about 0.5 inches wide and about 2-3 inches long.

In one embodiment, the tag 412 and the labels 472, 472b are all used as part of a mother-father-baby identification system at a hospital. The sheet 470 is passed through a printer and the labels 472, 472b and the tag 412 (at the identification portion 416 and/or wings 420, 422) are printed with identification information for the mother, father, and/or baby. The labels 472 can be printed with the mother's and baby's names and their doctor's name, and the labels are then removed from the sheet 470 and adhered to medical charts, files, prescriptions, bills, etc. The tag 412 is printed with the mother's and baby's names and is removed and looped around the baby's wristband, as described above, to provide identification information without curving around the small circumference of the baby's wrist (or ankle). The labels 472b can be removed and adhered to the mother's, father's, and/or baby's wristbands. For example, the two larger labels 472b can be adhered to the mother's and father's wristbands, respectively, and the two smaller labels 472b can be adhered to the baby's wristband and ankleband (as infants are often provided with both a wristband and ankleband at the hospital). These small labels 472 provide a small area for information on the baby's wristband, while the tag 412 provides additional area for identification information for the baby. Of course, not all of these labels are necessarily used in any given application.

An identification system 510 according to another embodiment of the invention is shown in FIG. 9. A sheet 570 includes a paper portion 570a and a polyester portion 570b, divided by a perforation 588. The paper portion 570a includes several labels 572. The fabric portion 570b includes a tag 512 and additional labels 572a, 572b. In this embodiment, as compared to FIG. 8, additional labels 572a are included in the polyester portion 570b.

In one embodiment, the sheet 570 is used as part of mother-father-baby identification system at a hospital. The paper labels 572 are printed with identification information and attached to medical charts, prescriptions, bills, etc. The polyester labels 572b are printed with identification information and attached to the mother's, father's, and baby's wristbands as described above. The polyester labels 572a are used to label breastmilk and any other samples or medications that are placed into a cooler or refrigerator. Labels on these items are exposed to moisture and condensation due to the cooler. Paper labels may disintegrate with this exposure. However, the polyester labels 572a can withstand the moisture and remain securely adhered to the sample. Accordingly, the polyester labels 572a can be used as part of the overall mother-father-baby identification system including identifying samples stored in a cooler or exposed to condensation or liquid (including breastmilk, blood samples, centrifuged samples, and other lab work and medications). The portion 570b may be a synthetic moisture-resistant material such as polyester.

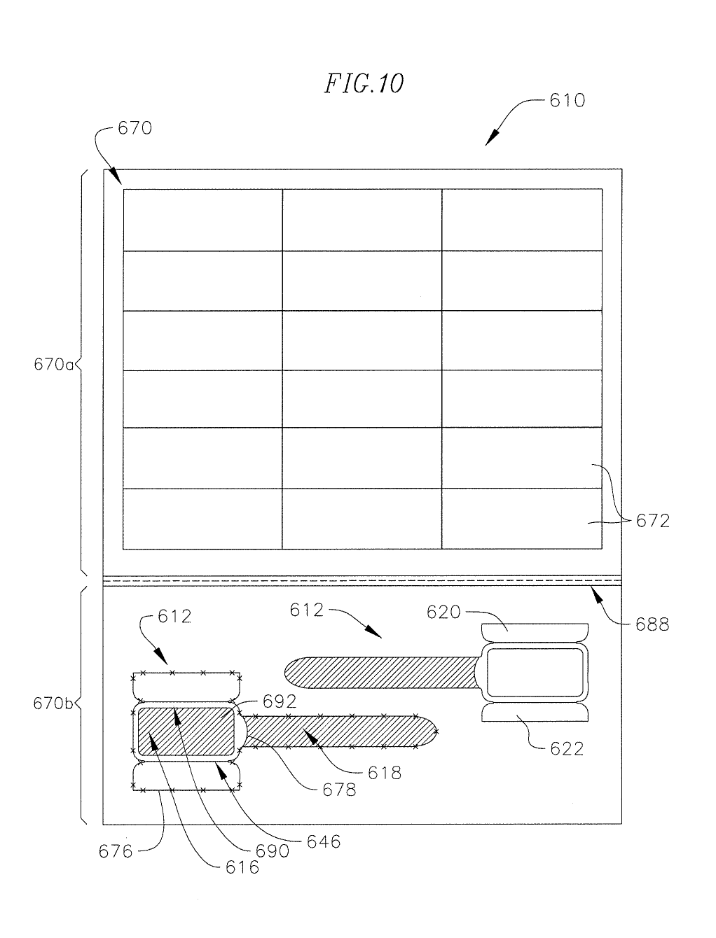

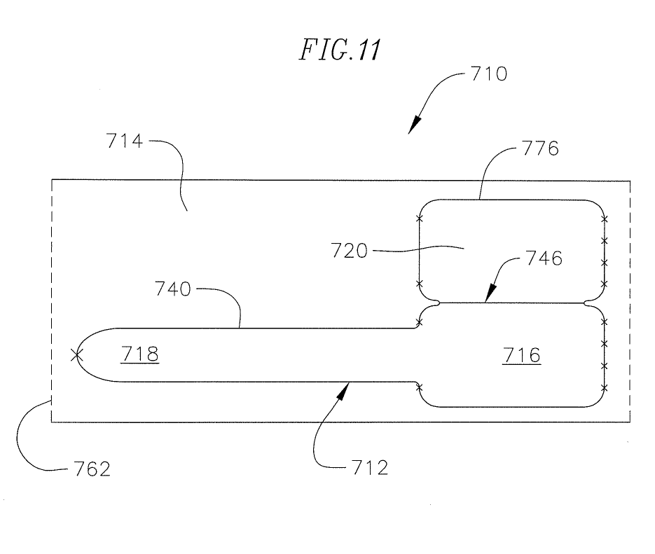

An identification system 610 according to another embodiment of the invention is shown in FIG. 10. A sheet 670 includes a paper portion 670a and a polyester portion 670b, divided by a perforation 688 that extends across the narrow dimension of the paper, rather than the longer dimension as in FIG. 8-9. The sheet 670 includes two tags 612 in the polyester portion 670b. The tags may be the same size, as shown, or they can be different sizes. In one embodiment, the tags are printed with identification information and attached to a baby's wristband and ankleband to identify the baby. The sheet 670 also includes paper labels 672.

The sheets 470, 570, and 670 are designed to pass through a laser or inkjet printer to print information on the labels and tags. In one embodiment, the sheets are 8.5 inches wide and 11 inches in length.

FIG. 8-10 show examples of different ways that a sheet can be arranged with labels, tags, paper, and polyester, to show that the sheet can be arranged in various different ways, and the features can take various sizes and positions, within the spirit and scope of the invention. It should be noted that although FIGS. 8-10 are top views of the sheet, certain portions of the bottom surface are shown for illustrative purposes (such as the cuts in the bottom layer forming the removable cover piece).

While a tag with two wings is discussed above and shown in many of the figures, in an alternative embodiment, only one wing is provided. An identification system 710 including a tag 712 with one wing 720 is shown in FIG. 11. In this embodiment, the width of the wing is extended so that the wing 720 extends over the free end of the strap 718 when the strap is looped under the identification portion 716. The wing should have a sufficient width to cover all of the exposed adhesive on the bottom surface of the tag when the cover piece is removed. A single removable cover piece may be provided, to be removed in one piece, with the exposed adhesive being completely covered by the single wing. The cover piece may include a curved end underlying the strap portion, a portion underlying the wing, and a bridge connecting these portions, so that the cover piece is all one continuous piece, as described above. The wing folds over at fold line 746, between the wing and the identification portion. The tag is formed on a tape 714, which in one embodiment is a direct thermal tape for printing by a thermal printer. The tag 712 can be separated from other tags at tear line 762, and then removed from the tape 714 along outline 740. The identification portion 716 and wing 720 have rounded corners for patient comfort.

Although limited embodiments of the identification system have been specifically described and illustrated herein, many modifications and variations will be apparent to those skilled in the art. For example, the one or more wings may take on other shapes and sizes, and different combinations of tags, labels, adhesives, and materials may be used. Accordingly, it is to be understood that the identification system constructed according to principles of this invention may be embodied other than as specifically described herein. The invention is also defined in the following claims.

What is claimed is:

1. An identification tag comprising:
an identification portion having first and second opposite sides and first and second opposite ends;
a strap portion extending from the first end of the identification portion;
a wing extending from the first side of the identification portion;
a curved notch forming curved corners where the wing meets the identification portion; and
a removable cover piece underlying at least a portion of the wing.

2. The tag of claim 1, wherein the strap portion has a length that is less than about 3 inches.

3. The tag of claim 1, wherein the tag comprises a top layer, a bottom layer, and an adhesive layer between the top and bottom layers.

4. The tag of claim 3 wherein the bottom layer comprises a section that remains adhered to the top layer in the identification portion after the cover piece is removed.

5. The tag of claim 3, further comprising a fold line in the top layer of the tag between the wing and the identification portion.

6. The tag of claim 3, wherein the bottom layer comprises a plurality of cuts forming the removable cover piece, and wherein the plurality of cuts includes a first cut in the strap portion and a second cut in the identification portion.

7. The tag of claim 6, wherein the second cut is a U-shaped cut separating the removable cover piece from a section of the bottom layer that remains adhered to the top layer when the cover piece is removed.

8. The tag of claim 6, wherein the plurality of cuts further comprises a third cut in the identification portion forming a first corner portion of the bottom layer that remains adhered to the top layer when the cover piece is removed.

9. The tag of claim 3, wherein the top layer comprises polyester, and the bottom layer comprises a liner with a release coating.

10. The tag of claim 1, further comprising a second wing extending from the second side of the identification portion, and wherein the cover piece underlies both the first and second wings, a portion of the identification portion, and a portion of the strap portion, and wherein the cover piece is continuous for removal as one piece.

11. The tag of claim 10, wherein the cover piece comprises a bridge connecting each wing to the strap portion.

12. The tag of claim 1, further comprising a second wing extending from the second side of the identification portion, and further comprising information printed on the first and second wings.

13. The tag of claim 12, wherein the information printed on the first wing identifies a mother, and the information printed on the second wing identifies a baby.

14. The tag of claim 1, further comprising a second wing extending from the second side of the identification portion, and wherein each of the first and second wings has a straight edge opposite the identification portion.

15. The tag of claim 14, wherein the straight edges meet when the wings are folded over the identification portion.

16. The tag of claim 3, further comprising a second wing extending from the second side of the identification portion, and wherein each wing comprises a curved corner where the wing meets the identification portion and a straight edge opposite the identificatin portion, and wherein the cover piece is a continuous piece underlying a portion of the strap portion, a portion of the identification portion, and a portion of each wing, and wherein the strap portion has a length that is less than about 3 inches.

17. An identification system comprising:
a direct thermal tape comprising a top layer, a bottom layer, and an adhesive between the top and bottom layers,
wherein the tape comprises one or more cuts outlining an identification tag having an identification portion, a strap portion, and a wing extending from the identification portion, wherein the strap portion has a length less than about three inches, and
wherein the top layer comprises a fold line between the wing and the identification portion, and
wherein the bottom layer comprises one or more cuts defining a removable cover piece underlying the wing.

18. The system of claim 17, wherein the one or more cuts in the bottom layer comprise a first cut in the strap portion and a second cut in the identification portion.

19. The system of claim 18, wherein the fold line is offset from the second cut.

20. The system of claim 19, wherein the cover piece comprises a strap section underlying the strap portion, a wing section underlying the wing, and a bridge connecting the strap section to the wing section.

21. The system of claim 17, wherein the one or more cuts in the tape outlining the tag comprise a first cut extending through both the top layer and the bottom layer of the tape, and a second cut extending into but not through at least one of the top or the bottom layers.

22. An identification system comprising:
a sheet having a top layer, a bottom layer, a release coating on at least a portion of the bottom layer, and an adhesive between the top layer and the bottom layer, the sheet being sized to pass through a printer;
an identification tag formed in the sheet and comprising an identification portion, a strap portion, and first and second wings on opposite sides of the identification portion; and
a plurality of labels carried by the sheet,
wherein the sheet comprises a first portion wherein the top layer comprises paper and a second portion wherein the top layer comprises polyester, and wherein the tag is formed in the second portion.

23. The identification system of claim 22, wherein the plurality of labels comprises a first label formed in the first portion of the sheet and a second label formed in the second portion of the sheet.

24. The identification system of claim 23, wherein the adhesive is absent along at least a portion of the strap portion.

25. The identification system of claim 24, wherein the identification portion comprises a bonded portion where the release coating is absent and the top and bottom layers both contact the adhesive.

26. The identification system of claim 25, wherein the bonded portion of the identification portion is located inside a border of the identification portion where the release coating is present.

27. The identification system of claim 22, wherein the strap portion is less than about 3 inches in length.

28. An identification system comprising:
a wristband; and
a tag comprising an identification portion carrying identification information, a strap portion extending from an end of the identification portion, and first and second wings extending from first and second opposite sides of the identification portion,
wherein the strap portion of the tag is looped around the wristband and behind the identification portion, and wherein the first and second wings are folded over and secured to the identification portion with the strap portion secured between the folded wings and the identification portion.

29. The identification system of claim 28, wherein the strap portion is secured into a loop by a first area of exposed adhesive on at least a portion of the identification portion, and wherein the first and second wings are secured to the identification portion by second and third areas of exposed adhesive on at least a portion of each wing, respectively, and wherein the first area of exposed adhesive contacts the second and third areas to form a continuous area of exposed adhesive.

30. The identification system of claim 28, wherein the strap portion has a length less than about 3 inches.

31. A method of identifying patient information in a medical setting, comprising:
providing a tag on a sheet or tape, the tag having an identification portion, a strap portion extending from an end of the identification portion, and a wing extending from a side of the identification portion;
printing information on the identification portion;
separating the tag from the sheet or tape; and
securing the tag to a patient wristband in a loop.

32. The method of claim 31, wherein securing the tag in a loop comprises:
removing a removable cover piece underlying the wing;
looping the strap portion around the patient wristband and under the identification portion; and
folding the wing and adhering it to the identification portion with the strap secured in a loop between the wing and the identification portion.

33. The method of claim 31, wherein providing the tag comprises providing the tag on a sheet having a plurality of polyester labels, and wherein the method further comprises printing information on one of the polyester labels and securing the printed polyester label to a refrigerated item.
